(19) United States
(12) Patent Application Publication
(10) Pub. No.: US 2009/0252763 A1
(43) Pub. Date: Oct. 8, 2009

(54) BIOLOGICAL ADJUVANTS

(75) Inventor: Ian Stuart Pardoe, Birmingham (GB)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: Henderson Morley PLC, Birmingham (GB)

(21) Appl. No.: 12/121,119

(22) Filed: May 15, 2008

(30) Foreign Application Priority Data
Apr. 8, 2008 (GB) 0806344.8

(51) Int. Cl. A61K 39/00; C12N 15/87; A61P 37/00

(52) U.S. Cl. 424/229.1; 435/456; 424/204.1; 424/278.1

(57) ABSTRACT

A method of enhancing uptake of DNA by a host cell line comprises introducing the DNA into the host cell and contacting the host cell with non-infectious viral particles. The non-infectious particles may be L-particle or PREPS (pre-viral DNA replication envelope particles), for example, those derived from alpha herpesvirus.

The present invention relates to adjuvants for use with a vaccine and, in particular, to biological adjuvants.

Further, the invention relates to adjuvants for naked DNA vaccines and to such vaccines as well as to methods of their manufacture.

### BACKGROUND OF THE INVENTION

Adjuvants are substances which, when added to a vaccine, improve the immune response so that less vaccine is needed to produce a protective response.

From very early in their replication cycle, viruses such as herpes viruses produce two different types of virus particles known as heavy (H) and light (L) particles. H-particles are able to spread and initiate new infections whereas L-particles lack viral DNA and its associated proteins and are, therefore, non-infectious.

Herpes Simplex Virus 1 (HSV-1) has been shown to produce L-particles. Such particles contain most, if not all of the envelope and tegument proteins of the H-particle (Szilagyi & Cunningham, 1991).

L-particles have been shown to enhance infectivity of HSV-1 and related viruses due to their incorporation of proteins capable of inducing the lytic cycle from otherwise non-replicating molecules within infected cells (Dargan & Subak-Sharpe, 1997).

It has also been demonstrated that adsorption and penetration of HSV-1 may be adversely affected by too high a proportion of L-particles per cell to be infected; more than 10,000 L-particles per cell has an adverse effect on penetration of HSV-1, while more than 1,000 L-particles per cell has an inhibitory effect on adsorption (Dargan & Subak-Sharpe, 1997).

Other non-infectious particles derivable from HSV-1 include PREPS (pre-viral DNA replication envelope particles), which can be made by blocking DNA replication during HSV-1 infection (Dargan, Patel & Subak-Sharpe, 1995)

It has now surprisingly been found that both PREPS and L-particles can act to increase uptake of DNA into cells and in particular mammalian cells. The DNA which may be enhanced in this manner is not limited to viral DNA such as HSV-1 viral DNA.

### SUMMARY OF THE INVENTION

In accordance with a first aspect of the invention, there is provided a method of enhancing uptake of DNA by a host cell, comprising introducing the DNA into the host cell and characterised in that the method further comprises contacting the host cell with non-infectious viral particles, preferably L-particles and/or PREPS.

The inclusion of such non-infectious particles in e.g. the cell culture medium can greatly increase uptake of the DNA by a host cell.

The non-infectious particles may be introduced separately from or in combination with the DNA.

The DNA may be a simple DNA strand, naked DNA or a DNA construct such as a plasmid.

In accordance with another aspect of the invention, there is provided a method of making a DNA vaccine, characterised in that the method comprises incorporating non-infectious viral particles, preferably L-particles and/or PREPS, into the vaccine.

In accordance with another aspect of the invention, there is provided a DNA vaccine characterised in that it includes non-infectious viral particles, preferably L-particles and/or PREPS.

The vaccine may be a naked DNA vaccine.

Yet a further aspect of the invention provides an adjuvant for use with a DNA vaccine, characterised in that it comprises non-infectious viral particles, preferably L-particles and/or PREPS.

A final aspect of the invention provides the use of non-infectious viral particles, preferably L-particles and/or PREPS, in the preparation of an adjuvant.

In a preferred configuration of the invention in its various aspects described above, the non-infectious viral particles, e.g. L-particles and/or PREPS, are derived from alpha herpesviruses, for example HSV-1, HSV-2, EBV, pseudo rabies, VZV.

### BRIEF DESCRIPTION OF SEVERAL VIEWS OF THE DRAWINGS

In order that the invention may be more readily understood, more easily appreciated and readily carried into effect by those skilled in the art, it will now be explained purely by way of non-limiting example with reference to the accompanying drawings where:—

FIG. 1 shows a typical infectious virus particle (virion);

FIG. 2 shows a typical L-particle; and

FIG. 3 shows the effect of HSV-1 derived L-particles and PREPS on the uptake of a pDS_GFP-XB plasmid by BHK cells; and

FIGS. 4A and 4B show photographs of cells exposed to plasmid alone (FIG. 4A) and the PREPS and plasmid (FIG. 4B).

### DETAILED DESCRIPTION OF THE PREFERRED EMBODIMENTS

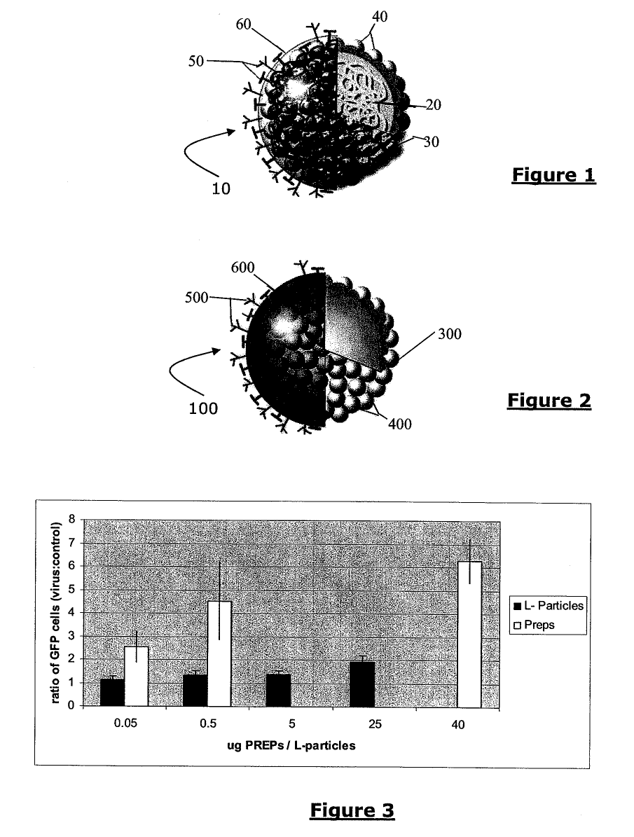

Referring now to FIG. 1, a virion 10 comprises a complement of DNA strands 20 surrounded by a capsid 30 studded with tegument proteins 40 and glycoproteins 50, referred to as surface proteins. The capsid 30 may itself be surrounded by a membrane 60.

FIG. 2 shows a typical L-particle 100. These are straight forward to manufacture and are generally produced in equal numbers to the virions. L-particles are non-infectious as the capsid 300 contains no DNA, but has a full complement of tegument proteins 400 and glycoproteins 500 and may be surrounded by a membrane 600.

L-particles can be produced by engineered viruses which thus makes them able to express non-native proteins. L-particles may be made using the teaching of U.S. Pat. No. 5,384,122, the entire disclosure of which is incorporated therein by reference.

The present inventors have also found that L-particles and PREPS can increase uptake and expression of non-native DNA in host cells. PREPS may be made using the teaching of U.S. Pat. No. 5,994,116, the entire disclosure of which is incorporated herein by reference.

Recombinant cell lines are commonly created by the transfection of novel DNA into the cells using plasmids which carry the gene to be expressed, together with a marker gene which allows the selection of these cells which are expressing the plasmid DNA.

The plasmid pDS_GFP-XB is an expression system suitable for use in mammalian cells. Successful uptake of the plasmid results in transfected cells expressing enhanced green fluorescent protein (eGFP).

To determine whether L-particles and PREPS could increase the uptake of plasmid DNA into mammalian cells, transfection of BHK cells with the above plasmid was carried out in accordance with the following protocol:
1. L-particles and PREPS were prepared from HSV-1 according to known methods.
2. BHK cells were grown to confluence in 6-well culture dishes and washed twice. Lipofectin (10 μl) was mixed with 100 μl of cell culture medium and incubated at room temperature for 45 minutes.
3. 0.4 μg of pDS_GFP-XB was then added to the lipofectin-cell culture medium and incubated at room temperature for a further 15 minutes.
4. The L-particles were diluted in 1 ml of cell culture medium to give concentrations of 0.05, 0.5, 5 and 25 μg/ml.
5. The PREPS were diluted in 1 ml of cell culture medium to give concentrations of 0.05, 0.5 and 40 μg/ml.
6. The concentrations from step (4) and step (5) were then separately added to the pDS_GFP-XB and lipofectin solution from step (3) and mixed.
7. Each concentration from step (6) was then separately added to the washed BHK cells and incubated at 37° C. for one hour. The treatments were set up in triplicate and included a control well in which transfection of the pDS_GFP-XB was performed in the absence of L-particles or PREPS.
8. The cells were then washed twice in cell culture medium and overlaid with maintenance medium, before being incubated in a C0₂-gassed incubator at 37° C. for 2 days.
9. Fluorescent cells in each well were counted by microscopy and the number of fluorescent cells counted in the cells exposed to L-particles or PREPS was normalised against the number of fluorescent cells counted in the control wells, and is represented in FIG. 3, wherein the values represent the ratio of the number of fluorescent cells seen in treated wells compared with the untreated transfection control wells.

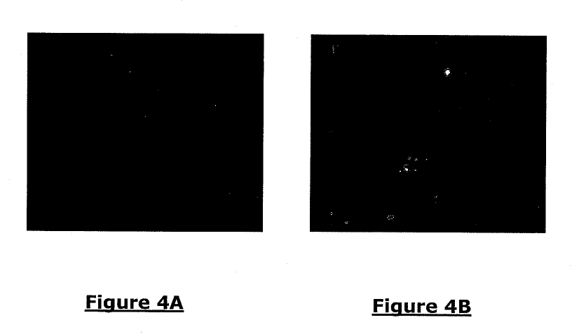

The results, as shown in FIGS. 3 to 4B, show a clear dose response, with the number of GFP expressing cells increasing by comparison with the control in proportion to the concentration of L-particles or PREPS added.

As will be understood from FIG. 3, cells exposed to PREPS showed a greater increase in the uptake of plasmid, while L-particles gave a smaller, but still statistically significant, increase in uptake of the plasmid.

While the example demonstrates expression of pDS_GFP-XB by BHK cells and the use of L-particles and PREPS derived from HSV-1, the skilled addressee can readily adapt the teachings herein toward other genes, cells and virally-derived L-particles and PREPS to obtain significant enhancement of uptake of DNA by mammalian cells. In particular, the DNA which may be thus enhanced is not limited to viral DNA, as demonstrated by the above example.

Without further elaboration of the foregoing will so fully illustrate our invention that others may, by applying current or future knowledge, adapt the same for use under various conditions of service.

1. A method of enhancing uptake of DNA by a host cell line, comprising introducing the DNA into the host cell and wherein the method further comprises contacting the host cell with non-infectious viral particles.

2. A method as claimed in claim 1, wherein the introducing and contacting steps are performed sequentially.

3. A method as claimed in claim 1, wherein the introducing and contacting steps are performed simultaneously.

4. A method as claimed in claim 1, wherein the non-infectious viral particles are L-particles and/or PREPS

5. A method as claimed in claim 4, wherein the L-particles and/or PREPS are derived from alpha herpesviruses.

6. A method as claimed in claim 1, wherein the DNA is a plasmid.

7. A method of making a DNA vaccine, wherein the method comprises introducing non-infectious viral particles into the vaccine.

8. A method as claimed in claim 7, wherein the non-infectious viral particles are L-particles and/or PREPS.

9. A method as claimed in claim 8, wherein the L-particles and/or PREPS are derived from HSV-1.

10. A method as claimed in claim 7, wherein the vaccine is a naked DNA vaccine.

11. A DNA vaccine comprising non-infectious viral particles.

12. A DNA vaccine as claimed in claim 11, wherein it is a naked DNA vaccine.

13. A DNA vaccine as claimed in claim 11, wherein the non-infectious viral particles are L-particles and/or PREPS.

14. A DNA vaccine as claimed in claim 13, wherein the L-particles and/or PREPS are derived from HSV-1.

15. An adjuvant for use with a DNA vaccine, wherein the adjuvant comprises non-infectious viral particles.

16. An adjuvant as claimed in claim 15, wherein the non-infectious viral particles are L-particles and/or PREPS.

17. An adjuvant as claimed in claim 16, wherein the L-particles and/or PREPS are derived from HSV-1.

18. Use of L-particles and/or PREPS in the preparation of an adjuvant.

19. Use of L-particles and/or PREPS as claimed in claim 18, characterised in that the L-particles and/or PREPS are derived from HSV-1.
